(19) United States
(12) Patent Application Publication
(10) Pub. No.: US 2013/0274650 A1
(43) Pub. Date: Oct. 17, 2013

(54) STABILIZED KETOROLAC COMPOSITIONS

(71) Applicant: LUITPOLD PHARMACEUTICALS, INC., Shirley, NY (US)

(72) Inventor: Richard P. Lawrence, Southold, NY (US)

(21) Appl. No.: 13/786,293

(22) Filed: Mar. 5, 2013

Related U.S. Application Data
(60) Provisional application No. 61/625,611, filed on Apr. 17, 2012.

(51) Int. Cl. A61K 31/407; A61M 11/02

(52) U.S. Cl. CPC A61K 31/407; A61M 11/02
USPC 604/24; 514/413

(57) ABSTRACT

Admixing carbon dioxide with an aqueous ketorolac solution is useful for increasing the stability of ketorolac in the carbon dioxide-admixed solution.

### CROSS-REFERENCE TO RELATED APPLICATION

This application claims the benefit under 35 U.S.C. §119 of U.S. Provisional Patent Application Ser. No. 61/625,611, filed on Apr. 17, 2012, which is incorporated hereby by reference in its entirety.

### FIELD OF THE INVENTION

Disclosed herein are stable, aqueous ketorolac compositions and delivery devices comprising such compositions. Also disclosed are processes for preparing such compositions, and methods of treatment employing such compositions and devices.

### BACKGROUND

U.S. patent application publication nos. U.S. 2010/0233099 and U.S. 2010/0233024 (each of which is incorporated herein in its entirety by reference) describe stabilized ketorolac compositions. In both cases, enhanced stability is achieved by modifying the gaseous components in the headspace of a closed vial containing the composition. Specifically, the gas in the headspace is modified by reducing the amount of molecular oxygen and replacing it with an inert gas. While such a modified gaseous environment in the headspace achieves prolonged shelf-life as compared to non-modified headspace, further enhancement in the shelf-life is desired.

### SUMMARY OF THE INVENTION

This invention is predicated, in part, on the discovery that modification of the aqueous solution rather than the gaseous headspace above the solution provides for a significant enhancement in the shelf-life of an aqueous ketorolac composition. Accordingly, in one of its composition aspects, this invention provides a stable, aqueous pharmaceutical composition comprising a ketorolac solution admixed with a ketorolac stabilizing amount of carbon dioxide.

The carbon dioxide can be admixed into the aqueous pharmaceutical composition in many forms such as gaseous carbon dioxide, dry ice (solid carbon dioxide), carbonate and bicarbonate salts, and the like. Without being limited to any theory, it is believed that the carbon dioxide in the aqueous solution will be present as carbonic acid (H₂CO₃) or a salt thereof such as a bicarbonate salt (e.g., NaHCO₃) wherein at least a ketorolac stabilizing amount of carbon dioxide is admixed in the composition.

As used in the composition, a sufficient amount of carbon dioxide is admixed with the solution so as to stabilize ketorolac from decomposing over time. Accordingly, as used herein, the term “stabilizing amount of carbon dioxide” is an amount of carbon dioxide that inhibits ketorolac decomposition so that the pharmaceutical composition exhibits a shelf-life of at least 6 months and preferably at least 2 years when stored under ambient conditions. In contrast to the prior embodiments using an inert gas, carbon dioxide is not inert as it reacts with the water or the aqueous solution to form carbonic acid or a salt thereof

In some embodiments, the aqueous ketorolac composition is saturated with carbon dioxide. In some embodiments, the aqueous ketorolac composition comprises a ketorolac stabilizing amount of carbon dioxide such as at least about 10%, 20%, 50%, 70% or 90% of the amount of saturated carbon dioxide, and ranges between any two of these values (including endpoints). The “amount of saturated carbon dioxide” as used herein refers to the amount of the carbon dioxide in an aqueous ketorolac composition described herein saturated with carbon dioxide at ambient temperatures, such as about 10° C., about 15° C., about 20° C., about 25° C., about 30° C., and ranges between any two of these values (including endpoints).

In some embodiments, the amount of carbon dioxide (or carbonic acid or a salt thereof) in the aqueous ketorolac composition is from about 1 mM to about 1 M. In some embodiments, the concentration is from about 10 mM to about 500 mM, or from about 50 mM to about 200 mM. In some embodiments, the concentration is about 5 mM, about 10 mM, about 15 mM, about 20 mM, about 50 mM, about 100 mM, about 200 mM, about 500 mM, about 700 mM, about 1 M, and ranges between any two of these values (including endpoints).

As used herein, “stable” or “stabilizing” refers to the ketorolac in the composition remaining substantially unchanged so that it is pharmaceutically suitable for its intended use during a relevant period of time. In one embodiment, when the composition is a medicament, the ketorolac in the composition is deemed stable if it meets the standard for being used as a medicament as per relevant regulatory agency, such as the United States Food and Drug Administration. Thus, a stable composition may comprise certain minor degree of degradation of one or more ingredients, so long as the degree of degradation is not significant enough to make it unusable for its intended use, e.g., fall below the standard for use as a medicament.

As used herein, the term “carbon dioxide” generically refers to any gas, liquid or solid that generates carbon dioxide in water where, as is well known, the carbon dioxide exists as its reaction product of H₂CO₃ or salts thereof. Examples of carbon dioxide include gaseous carbon dioxide, dry ice (solid carbon dioxide), and salts of carbon dioxide such as carbonate and bicarbonate salts. In one embodiment, these salts are pharmaceutically acceptable salts of carbon dioxide such as sodium, potassium, calcium, ammonium and substituted ammonium, and such other well known salts.

In a preferred embodiment, the aqueous composition is a sterile aqueous solution saturated with carbon dioxide. As used herein, the term “sterile” comports to that definition generally accepted in the art as being sterile. As used herein, “saturated” refers preferably to saturated under a certain temperature and a certain pressure. In a preferred embodiment, the aqueous ketorolac compositions are sterile and are provided in a device suitable for intranasal delivery.

In one preferred embodiment, the composition comprises from 12.5% w/v to about 38% w/v of ketorolac tromethamine. As used herein, “w/v” refers to grams of a substance per 100 mL solution of that substance. In another preferred embodiment, the composition comprises 15% or 30% w/v of ketorolac tromethamine. Where the vial contains highly concentrated, e.g., 12-38% or 15-35% w/v, ketorolac solution, storing the ketorolac composition admixed with carbon dioxide, allows the composition to be stored under ambient conditions for a sufficiently long period of time, such as for at least 6 months, or for at least two years, without significant degradation or decomposition. In another embodiment, the composition further comprises about 4% to about 10% w/v of lidocaine hydrochloride. In another embodiment, the composition further comprises one or more of a chelator, a preservative, an absorption promoter, a gelling polymer, and a co-solvent. In another embodiment, the composition is suitable for nasal administration.

In one of its device aspects, this invention provides a nasal spray device comprising the aqueous composition provided herein. As used herein, a “nasal spray device” is a device containing an exit tip which can be inserted into a patient's nostril to spray a defined amount of the solution into the nostril. In one embodiment, the spray device comprises a vial having a cap, wherein the cap seals the vial from gaseous exchange with the atmosphere and comprises a spray system that allows delivery of a therapeutic amount of ketorolac into the nostril or nostrils of the patient. Preferably, the vial has a volumetric space of no more than about 10 mL and contains up to about 5 mL of a solution within that space. More preferably, the volume of the solution is about half of the volume of the volumetric space. The solution comprises a stable aqueous, pharmaceutical ketorolac composition as provided herein. A dip tube attached to the spray system dips into the composition. A gaseous head space occupies the remainder of the vial.

In certain preferred embodiments, the head space comprises no more than about 10% v/v oxygen. Within this embodiment, the head space comprises an inert gas, carbon dioxide or a mixture thereof with no more than about 8% v/v oxygen; or more preferably no more than about 5% v/v oxygen. In some embodiments, the head space comprises between about 5% to about 10% v/v oxygen; or between about 3% to about 8% v/v oxygen; or between about 2% to about 5% v/v oxygen. In certain embodiments, the head space comprises an inert gas or carbon dioxide. In some embodiments, the inert gas is nitrogen or argon. However, it is most preferred that the headspace comprises at least a portion of carbon dioxide.

In one of its process aspects, this invention provides a process of preparing a stabilized composition comprising ketorolac, the process comprising contacting carbon dioxide with an aqueous ketorolac solution, such that at least a ketorolac stabilizing amount of carbon dioxide is admixed in the composition. In one embodiment, the carbon dioxide is gaseous carbon dioxide which is bubbled through the solution until the solution is saturated with carbon dioxide. In another embodiment, carbon dioxide can be introduced as a carbonate or a bicarbonate salt.

In one of its treatment aspects, this invention provides a method of treating pain and/or inflammation in a human subject, the method comprising administering a therapeutically effective amount of a stabilized aqueous ketorolac composition as provided herein. In one embodiment, administration is readily achieved by use of a nasal spray device.

### BRIEF DESCRIPTION OF THE DRAWING

FIG. 1 illustrates a cross-section view of a vial comprises a ketorolac composition having a concave or V-shaped inner bottom, and a dip tube attached to a spray system and dipping into the ketorolac composition, and ending proximate the bottom of the inner bottom.

### DETAILED DESCRIPTION OF THE INVENTION

### Definitions

This invention relates to stable, aqueous ketorolac compositions. However, before further describing this invention, the following terms are defined.

The term “about” when used before a numerical designation, e.g., pH, temperature, amount, concentration, and molecular weight, including a range, indicates approximations which may vary by (+) or (- ) 5%, 1% or 0.1%.

As used in the specification and claims, the singular form “a”, “an” and “the” include plural references unless the context clearly dictates otherwise. For example, the term “a pharmaceutically acceptable salt” includes a plurality of pharmaceutically acceptable salts, including mixtures thereof.

“Administering” or “administration of” an agent to a patient (and grammatical equivalents of this phrase) refers to both direct administration, including self-administration, and indirect administration, including the act of prescribing a drug. For example, as used herein, a physician who instructs a patient to self-administer a drug and/or provides a patient with a prescription for a drug is administering the drug to the patient.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace amount of elements of other ingredients and substantial method steps. Embodiments defined by each of these transition terms are within the scope of this invention.

“Ketorolac” refers to the chemical compound of 5-benzoyl-2,3-dihydro-1H-pyrrolizine-1-carboxylic acid which has the following Formula (I):

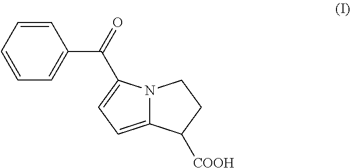 [SMILES: O=C(c1ccccc1)c1ccc2n1CCC2C(=O)O]

or a pharmaceutically acceptable salt thereof.

For purposes of this application, the name ketorolac encompasses individually or collectively the racemic mixture, a scalemic (or enantiomerically enriched) mixture, or optically active compound, and includes the pharmaceutically acceptable salts of ketorolac, particularly the tromethamine salt. As used herein, a racemic mixture of ketorolac is a mixture having equal amounts of the two enantiomers of Formula (I). A scalemic or enantiomerically enriched mixture of ketorolac is a mixture where the amount of one of the enantiomers of Formula (I) is larger than the other enantiomer. An optically active compound may include enantiomerically enriched or enantiomerically pure compound. In a preferred embodiment, the enantiomerically pure or enriched compound is the (S) enantiomer. Enantiomerically pure compound refers to ketorolac having more than 99%, e.g. 99.5%, or 99.9% of one of the enantiomers relative to the total amount of ketorolac.

“Lidocaine” refers to the chemical compound of 2-(diethylamino)-N-(2,6-dimethylphenyl)acetamide, which has the Formula (II):

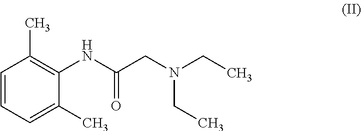 [SMILES: CCN(CC)CC(=O)Nc1c(C)cccc1C]

or a pharmaceutically acceptable salt thereof.

Many pharmaceutically acceptable salts of lidocaine are known. Non-limiting examples of such salts are lidocaine hydrochloride and lidocaine methanesulphonate. As used herein, the term “lidocaine” refers to the compound or any of its pharmaceutically acceptable salts, unless otherwise indicated.

“Pharmaceutically acceptable” refers to non-toxic and suitable for in vivo administration, preferably to humans, still more preferably by nasal administration.

“Pharmaceutical composition” refers to a pharmaceutically acceptable composition or formulation, and includes one or more active agents, such as ketorolac optionally with lidocaine, and one or more inactive components or excipients.

The term “pharmaceutically acceptable salt” refers to pharmaceutically acceptable salts derived from a variety of organic and inorganic counter ions well known in the art and include, by way of example only, when the molecule contains an acidic functionality, salts of organic or inorganic bases, such as sodium, potassium, calcium, magnesium, ammonium, tromethamine and tetraalkylammonium, and when the molecule contains a basic functionality, salts of organic or inorganic acids, such as hydrochloride, hydrobromide, tartrate, mesylate (methanesulfonate), acetate, maleate, and oxalate. Suitable salts include those described in P. Heinrich Stahl, Camille G. Wermuth (Eds.), Handbook of Pharmaceutical Salts Properties, Selection, and Use; 2002.

The term “subject” or “patient” is used interchangeably herein, and refers to a human.

“Therapeutically effective amount” of an agent refers to an amount of the agent that, when administered to a patient, will have the intended therapeutic effect, e.g., alleviation, amelioration, palliation, reduction, or elimination of one or more negative effects of the underlying disease in the patient. The full therapeutic effect does not necessarily occur by administration of one dose (or dosage), and may occur only after administration of a series of doses. Thus, an effective amount may be administered in one or more administrations.

“Treatment” or “treating” refers to any treatment of a disease or condition related to pain and/or inflammation in a subject, including one or more of: preventing or protecting against the disease or condition, that is, causing the relevant symptoms not to develop, for example, in a subject at risk of suffering from such a disease or condition, thereby substantially averting onset of the disease or condition; inhibiting the disease or condition, that is, arresting or suppressing the development of relevant symptoms; and relieving the disease or condition that is, causing the regression of relevant symptoms.

### Certain Embodiments

In some embodiments, the ketorolac solution or the composition provided herein comprises concentrations of ketorolac or a pharmaceutically acceptable salt thereof ranging from about 12.5 to 38% weight to volume (w/v), for example about 15%, 20%, 25%, 28%, 29%, 30%, 31%, 32%, 33%, 34%, 35%, 36%, 37%, or 38% w/v, based on the final solution or composition.

In some embodiments, the ketorolac solution or the composition provided herein comprises:
(a) about 12.5% w/v to about 38% w/v of ketorolac or a pharmaceutically acceptable salt thereof, and
(b) a pharmaceutically acceptable carrier.

In some embodiments, the ketorolac solution or the composition provided herein further comprises a local anesthetics, such as lidocaine or a pharmaceutically acceptable salt thereof ranging from about 4% to 10%, for example about 4%, 5%, 6%, 7%, 8%, 9%, or 10% w/v. Such compositions are disclosed in U.S. Patent Application Publication No. 2009/0042968, which is incorporated herein by reference in its entirety.

In some embodiments, the ketorolac solution or the composition provided herein comprises:
(a) about 12.5% w/v to about 38% w/v of ketorolac or a pharmaceutically acceptable salt thereof,
(b) about 4% w/v to about 10% w/v of lidocaine or a pharmaceutically acceptable salt thereof, and
(c) a pharmaceutically acceptable carrier.

In some embodiments, the pharmaceutically acceptable carrier is water.

In some embodiments, ketorolac is present as a racemic or scalemic mixture. In some embodiments, the pharmaceutically acceptable salt of ketorolac is ketorolac tromethamine. In some embodiments, the ketorolac solution or the composition provided herein comprises about 13% to 20% w/v of ketorolac tromethamine. In some embodiments, the ketorolac solution or the composition provided herein comprises about 15% w/v of ketorolac tromethamine. In some embodiments, the ketorolac solution or the composition provided herein comprises about 25% to 35% w/v of ketorolac tromethamine. In some embodiments, the ketorolac solution or the composition provided herein comprises about 28% to 32% w/v of ketorolac tromethamine. In some embodiments, the ketorolac solution or the composition provided herein comprises about 30% w/v of ketorolac tromethamine.

In some embodiments, the ketorolac solution or the composition provided herein comprises lidocaine hydrochloride. In some embodiments, the solution or the composition comprises about 5-6% lidocaine hydrochloride. In some embodiments, the solution or the composition comprises about 6% lidocaine hydrochloride.

In some embodiments, the ketorolac solution or the composition provided herein further comprises a chelator, i.e. a substance that binds primarily di- or tri valent metallic ions (e.g. calcium) that might interfere with the stability or activity of the active ingredient. Chelators are known to those of skill in the art by referring to the recent edition of “Remington's Pharmaceutical Sciences.” A preferred chelator is sodium ethylenediamine tetraacetic acid (sodium EDTA), USP. In some embodiments, the chelator is disodium edetate.

In some embodiments, the pH of the ketorolac solution or the composition provided herein is about 4.5 to 8. In some embodiments, the pH is about 4.8 to 7.5. In some embodiments, the pH is about 7.2.

In some embodiments, the pH is adjusted by a pharmaceutically acceptable base. In some embodiments, the pharmaceutically acceptable base is sodium hydroxide.

A pharmaceutically acceptable buffer may be present in order to create optimum pH conditions for both product stability and tolerance (pH range about 4 to about 8; preferably about 6.0 to 7.5). Suitable buffers include without limitation tris (tromethamine) buffer, phosphate buffer, etc. Preferably, potassium phosphate NF is used to adjust the pH to 7.2. In some embodiments, the composition comprises up to about 2% of a phosphate buffer, such as potassium phosphate monobasic, potassium phosphate dibasic. In some embodiments, the composition comprises about 0.6 to 0.8% w/v of potassium phosphate monobasic. In some embodiments, the composition comprises about 0.68% w/v of potassium phosphate monobasic.

In some embodiments, the composition provided herein is a sprayable liquid.

In some embodiments, the ketorolac solution or the composition provided herein comprises:
(a) about 15% w/v of racemic ketorolac tromethamine,
(b) about 0.01% w/v to about 0.1% w/v of disodium edetate,
(c) about 0.68% w/v of potassium phosphate monobasic,
(d) sodium hydroxide to adjust the pH to 7.2, and
(e) water to 100% w/v.

In some embodiments, the ketorolac solution or the composition provided herein comprises:
(a) about 15% w/v of racemic ketorolac tromethamine,
(b) about 5% w/v to about 6% w/v lidocaine hydrochloride,
(c) about 0.01% w/v to about 0.1% w/v of disodium edetate,
(d) about 0.68% w/v of potassium phosphate monobasic,
(e) sodium hydroxide to adjust the pH to 7.2, and
(f) water to 100% w/v.

In some embodiments, the ketorolac solution or the composition provided herein comprises:
(a) about 30% w/v of racemic ketorolac tromethamine,
(b) about 0.01% w/v to about 0.1% w/v of disodium edetate,
(c) about 0.68% w/v of potassium phosphate monobasic,
(d) sodium hydroxide to adjust the pH to 7.2, and
(e) water to 100% w/v.

In some embodiments, the ketorolac solution or the composition provided herein comprises:
(a) about 30% w/v of racemic ketorolac tromethamine,
(b) about 5% w/v to about 6% w/v lidocaine hydrochloride,
(c) about 0.01% w/v to about 0.1% w/v of disodium edetate,
(d) about 0.68% w/v of potassium phosphate monobasic,
(e) sodium hydroxide to adjust the pH to 7.2, and
(f) water to 100% w/v.

The ketorolac solution or the composition provided herein optionally comprises one or more pharmaceutically acceptable excipients such as a diluent. A preferred diluent for the compositions is water, and other excipients may be added as needed as will be apparent to the skilled artisan.

In addition to aqueous, oil or gel diluents, other diluents which may be used in the composition comprise solvent systems containing ethyl alcohol, isopropyl alcohol, propylene glycol, polyethylene glycol, mixtures thereof or mixtures of one or more of the foregoing with water.

Other excipients include chemical enhancers such as absorption promoters. These include fatty acids, bile acid salts and other surfactants, fusidic acid, lysophosphatides, cyclic peptide antibiotics, preservatives, carboxylic acids (ascorbic acid, amino acids), glycyrrhetinic acid, o-acylcarnitine. Preferred promoters are diisopropyladipate, POE(9) lauryl alcohol, sodium glycocholate and lysophosphatidyl choline which proved to be particularly active.

If present, excipients such as oil, gel, chemical enhancers, including abosorption promoters, etc. should be in an amount that does not adversely affect the homogeneity and sprayability of the solution.

The ketorolac solution or the composition provided herein can also contain a compatible preservative that ensures the microbiological stability of the active ingredient. Suitable preservatives include without limitation, methyl paraoxybenzoate (methyl paraben), propyl paraoxybenzoate (propyl paraben), sodium benzoate, benzyl alcohol, and chlorobutanol. In one embodiment, the intranasal ketorolac formulation does not contain a preservative.

Illustrative compositions may contain the following ingredients and amounts (w/v) in addition to ketorolac, lidocaine and water.

| Ingredient | Broad Range (%) | Preferred Range (%) |
|---|---|---|
| Chelator (1) | 0.001-1 | 0.01-0.1 |
| Preservative (2) | 0-2 | 0-0.25 |
| Absorption promoter (3) | 0-10 | 0-10 |
| Gelling polymer (4) | 0-5 | 0-3 |
| Co-solvent (5) | 0-99 | 0 |

(1) E.g., sodium EDTA
(2) E.g., methyl paraoxybenzoate or propyl paraoxybenzoate or mixtures thereof
(3) E.g., sodium glycocholate
(4) E.g., sodium CMC
(5) E.g., glycerol

Methods for preparing vials and nasal spray devices containing ketorolac compositions are reported (see, U.S. 2010/0233099 and U.S. 2010/0233024, each supra) and can be modified by appropriate substitution of the pharmaceutical compositions to prepare the vials and nasal delivery devices of this invention.

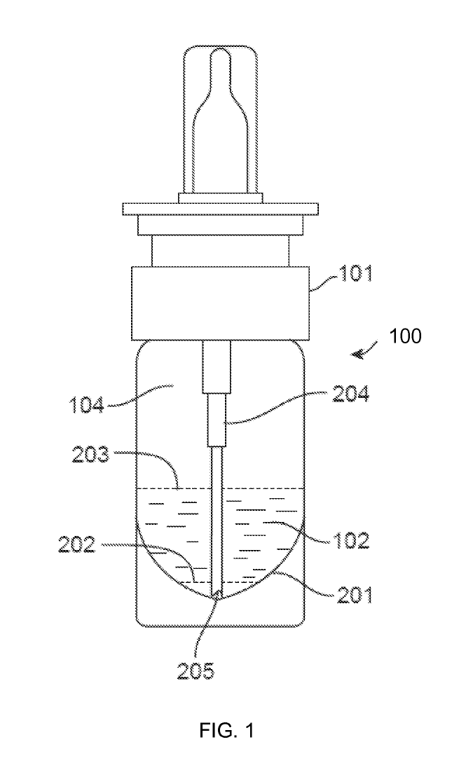

In some embodiments, the vial comprises a concave or V-shaped (i.e. conical) inner bottom, and a dip tube attached to a spray system and dipping into the ketorolac composition contained in the vial, and ending proximate (such as no more than about 2 mm or 1 mm from) the bottom of the inner bottom. FIG. 1 illustrates an example of such a vial 100 comprising a concave or V-shaped inner bottom 201, a cap 101 having a spray system, and a dip tube 204. The dip tube 204 is attached to the spray system and dips into a ketorolac solution 102. The tip 205 of the dip tube ends proximate to the inner bottom 201. The vial further comprises a head space 104. The ketorolac solution in the vial has an initial amount shown by line 203, and when all is used by a patient, a residue amount shown by line 202, which is minimal due to the presence of the concave or V-shaped inner bottom 201.

In some embodiments, the nasal spray device is further equipped with a metering chamber to measure a desired amount of the composition to be sprayed into the patient's nasal passage. In one embodiment, the metering chamber is coupled with the spraying device so that a patient can simultaneously measure and spray a desired amount (e.g. a unit dose) of the composition. In one embodiment, the metering chamber is able to deliver a predetermined amount of about 50 to about 125 microliters of liquid. In one embodiment, the metering chamber is able to deliver from about 50 to about 100 microliters of liquid. In one embodiment, the metering chamber having different volume deliveries can be used. In one embodiment, the metering chamber is able to measure about 50 microliters of liquid. In one embodiment, the metering chamber is able to measure about 100 microliters of liquid. Appropriate vials and spray devices with or without a metering chamber are available to one of skill in the art by referring to “Remington's Pharmaceutical Sciences.” One source for such vessels is Ing. Erich Pfeiffer GmbH, Radolfzell, Germany. Another source is Valois, 50 avenue de l'Europe, 78164 MARLY-LE-ROI, France.

In another aspect, the vial or nasal spray device of this invention is in combination with labeling instructions for use in treating a pain or inflammation in a human subject. In some embodiments, the pain is the result of a trauma inflicted on the subject. In some embodiments, the pain is the result of a medical operation performed on the subject. In some embodiments, the pain is pathological. In some embodiments, the pain is neuropathic. In some embodiments, the pain is migraine or other headache pain.

The following examples are provided to illustrate certain aspects of the present invention and to aid those of skill in the art in practicing the invention. These examples are in no way to be considered to limit the scope of the invention.

### EXAMPLES

Accelerated stability of ketorolac solutions were determined using carbon dioxide (lot LA111301) and argon (lot LA102771) in closed systems (Sprix-vials sealed with Teflon stoppers). Carbon dioxide was, surprisingly, as effective as Argon in stabilizing the ketorolac solution. Based on the results of the accelerated stability, it is contemplated that carbon dioxide can stabilize ketorolac solution in a sealed vial for about 1 year at room temperature. For example, when stored for about a year, the 1-keto degradant:

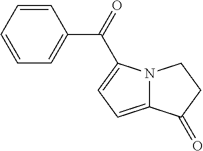 [SMILES: O=C1CCn2c1ccc2C(=O)c1ccccc1]

is produced in an amount not more than 1.5% of ketorolac, and in certain instances, the total degradants produced are not more than 2.0% of ketorolac.

When accelerated stability of ketorolac solutions contained in vials stoppered with spray tops were determined, carbon dioxide was as effective in stabilizing the solution as argon. In these vials, the solution can release carbon dioxide above the solution into the vials's headspace, and protect the solution from the decomposing effect of any oxygen present in the headspace above the carbon dioxide layer.

1. A stable, aqueous pharmaceutical composition comprising a ketorolac solution admixed with at least a ketorolac stabilizing amount of carbon dioxide.

2. The composition of claim 1, wherein the ketorolac stabilizing amount of carbon dioxide is at least about 10% of the amount of saturated carbon dioxide.

3. The composition of claim 1, which is saturated with carbon dioxide.

4. The composition of claim 1, wherein the ketorolac stabilizing amount of carbon dioxide is from about 1 mM to about 1 M.

5. The composition of claim 1, which is at a pH of about 4 to about 8.

6. The composition of claim 1, which comprises from 12.5% w/v to about 38% w/v of ketorolac tromethamine.

7. The composition of claim 1, which comprises 15% or 30% w/v of ketorolac tromethamine.

8. The composition of claim 1, which further comprises about 4% to about 10% w/v of lidocaine hydrochloride.

9. The composition of claim 1, which further comprises one or more of a chelator, a preservative, an absorption promoter, a gelling polymer, and a co-solvent.

10. The composition of claim 1, which is stored for up to 2 years.

11. The composition of claim 1, which is for intranasal administration.

12. A nasal spray device comprising a vial having a cap, wherein the vial has a volumetric space of no more than about 10 mL and contains up to about 5 mL of the stable aqueous pharmaceutical composition of claim 1, the cap seals the vial from gaseous exchange with the atmosphere and comprises a spray system that allows delivery of a therapeutically effective amount of ketorolac into the nostril or nostrils of a patient, a dip tube that is attached to the spray system and dips into the aqueous composition, and a head space occupies the remainder of the vial.

13. The nasal spray device of claim 12, wherein the vial comprises a concave or V-shaped inner bottom.

14. The nasal spray device of claim 12, wherein the head space comprises carbon dioxide and optionally an inert gas.

15. The nasal spray device of claim 1, wherein the head space comprises no more than about 10% v/v oxygen.

16. A method of treating pain and/or inflammation in a human subject in need thereof, the method comprising:
administering a therapeutically effective amount of the stable aqueous pharmaceutical composition of claim 1.

17. A process of preparing an aqueous composition comprising ketorolac, the process comprising contacting carbon dioxide with an aqueous ketorolac solution, such that at least a ketorolac stabilizing amount of carbon dioxide is admixed in the composition.

18. A method of treating pain and/or inflammation in a human subject in need thereof, the method comprising administering a therapeutically effective amount of the stable aqueous pharmaceutical composition of claim 1 from a nasal spray device, wherein the nasal spray device comprises a vial having a cap, wherein the vial comprises a volumetric space of no more than about 10 mL and contains up to about 5 mL of the stable aqueous pharmaceutical composition, the cap seals the vial from gaseous exchange with the atmosphere and comprises a spray system that allows delivery of the therapeutically effective amount of the stable aqueous pharmaceutical composition into a nostril of a patient, a dip tube that is attached to the spray system and dips into the aqueous composition, and a head space occupies the remainder of the vial.
